(19) United States
(12) Patent Application Publication
(10) Pub. No.: US 2026/0240535 A1
(43) Pub. Date: Aug. 20, 2026

(54) TISSUE ANCHOR SYSTEM AND METHODS OF DEPLOYING A TISSUE ANCHOR SYSTEM

(71) Applicants: Smith & Nephew, Inc., Memphis, TN (US); Smith & Nephew Asia Pacific Pte. Limited, SINGAPORE (SG)

(72) Inventors: Dennis P. Colleran, North Attlebor, MA (US); Nehal N. Patel, Boston, MA (US)

(73) Assignees: Smith & Nephew, Inc., Memphis, TN (US); Smith & Nephew Asia Pacific Pte. Limited, SINGAPORE (SG)

(21) Appl. No.: 19/529,391

(22) Filed: Feb. 4, 2026

Related U.S. Application Data
(60) Provisional application No. 63/760,821, filed on Feb. 20, 2025.

(51) Int. Cl. A61B 17/04

(52) U.S. Cl. CPC A61B 17/0401; A61B 2017/0409; A61B 2017/0414; A61B 2017/0464

(57) ABSTRACT

A tissue anchor system includes an anchor assembly including a first anchor, a second anchor, and a suture extending therebetween, and an inserter instrument including a penetrating member and a retainer positioned proximally of and laterally offset from the penetrating member. The penetrating member includes a lumen configured to removably receive the first anchor. The retainer includes a lumen configured to removably receive the second anchor. A longitudinal axis of the first anchor when disposed within the penetrating member is parallel to a longitudinal axis of the second anchor when disposed within the retainer. A method of deploying the anchor assembly includes advancing the penetrating member into tissue or bone until a distal shoulder of the inserter instrument abuts an outer surface of the tissue or bone, deploying the first anchor from the penetrating member, and deploying the second anchor from the retainer outside of the tissue or bone.

### CROSS-REFERENCE TO RELATED APPLICATIONS

This application claims the benefit of U.S. Provisional Patent Application Serial No. 63/760,821 filed on February 20, 2025, the disclosure of which is incorporated herein by reference.

### TECHNICAL FIELD

The present disclosure pertains generally to medical devices and methods of using medical devices. More particularly, the present disclosure relates to tissue anchor systems and methods of using the same.

### BACKGROUND

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear, or from overuse of the joint. In arthroscopic procedures, there are challenges to repair injuries such as, but not limited to, tying knots to close a tear, securing an implantable scaffold at an injury site, etc. Current techniques include the use of sutures or staples. Suturing may be time consuming and/or tedious. Stapling relies upon tissue quality for securement, and in some instances, the tissue quality is inadequate to properly hold the staple(s). There is an ongoing need for alternative devices and/or methods during an arthroscopic procedure in order to treat injuries to the rotator cuff, rotator cuff tendons, or other soft tissue or tendon injuries throughout a body.

### SUMMARY

This disclosure provides design, material, manufacturing method, and use alternatives for medical devices, including systems for delivering a medical implant along with bone and/or tissue anchors into an operative space.

In a first example, a tissue anchor system may comprise an anchor assembly including a first anchor, a second anchor, and a suture extending therebetween, and an inserter instrument including a penetrating member and a retainer positioned proximally of and laterally offset from the penetrating member. The penetrating member may include a lumen configured to removably receive the first anchor. The retainer may include a lumen configured to removably receive the second anchor.

In addition, or alternatively, to any example disclosed herein, the penetrating member includes a curved distal tip.

In addition, or alternatively, to any example disclosed herein, the penetrating member is configured to penetrate tissue.

In addition, or alternatively, to any example disclosed herein, the inserter instrument is configured to deploy the first anchor from the lumen of the penetrating member below an upper surface of the tissue.

In addition, or alternatively, to any example disclosed herein, the inserter instrument is configured to deploy the second anchor from the lumen of the retainer above an upper surface of the tissue.

In addition, or alternatively, to any example disclosed herein, the penetrating member is configured to penetrate a cortical shell of a bone.

In addition, or alternatively, to any example disclosed herein, the inserter instrument is configured to deploy the first anchor from the lumen of the penetrating member into cancellous bone disposed beneath the cortical shell.

In addition, or alternatively, to any example disclosed herein, the inserter instrument is configured to deploy the second anchor from the lumen of the retainer above an upper surface of the cortical shell.

In addition, or alternatively, to any example disclosed herein, a length of the suture extending between the first anchor and the second anchor is adjustable.

In addition, or alternatively, to any example disclosed herein, the length of the suture extending between the first anchor and the second anchor is adjustable via passing a first segment of the suture through a second segment of the suture.

In addition, or alternatively, to any example disclosed herein, the first anchor and the second anchor are each formed from a substantially rigid polymeric material.

In addition, or alternatively, to any example disclosed herein, the first anchor and the second anchor are each formed from a woven material or a braided material.

In addition, or alternatively, to any example disclosed herein, and in a second example, a tissue anchor system may comprise an anchor assembly including a first anchor, a second anchor, and a suture extending therebetween, and an inserter instrument including a penetrating member and a retainer positioned proximally of and laterally offset from the penetrating member. The first anchor may be removably disposed within a lumen of the penetrating member. The second anchor may be removably disposed within a lumen of the retainer. A longitudinal axis of the first anchor when disposed within the lumen of the penetrating member may be parallel to a longitudinal axis of the second anchor when disposed within the lumen of the retainer.

In addition, or alternatively, to any example disclosed herein, the first anchor is deployable from the lumen of the penetrating member into tissue or bone, and the second anchor is deployable from the lumen of the retainer outside of the tissue or bone.

In addition, or alternatively, to any example disclosed herein, the tissue anchor system may comprise an implantable scaffold configured to be deployed against an outer surface of the tissue or bone.

In addition, or alternatively, to any example disclosed herein, the second anchor is configured to secure an implantable scaffold against the outer surface of the tissue or bone.

In addition, or alternatively, to any example disclosed herein, a first end of the suture is coupled to the first anchor at a medial location between a first end of the first anchor and a second end of the first anchor, and a second end of the suture is coupled to the second anchor at a medial location between a first end of the second anchor and a second end of the second anchor.

In addition, or alternatively, to any example disclosed herein, the first anchor is deployable from the lumen of the penetrator in a direction non-parallel to the longitudinal axis of the first anchor when disposed within the lumen of the penetrating member.

In addition, or alternatively, to any example disclosed herein, and in a third example, a method of deploying an anchor assembly may comprise: advancing a penetrating member of an inserter instrument into tissue or bone until a distal shoulder of the inserter instrument abuts an outer surface of the tissue or bone; deploying a first anchor from a lumen of the penetrating member; and deploying a second anchor from a lumen of a retainer of the inserter instrument outside of the tissue or bone, wherein the lumen of the retainer is disposed proximally of and laterally offset from the penetrating member. The first anchor may be coupled to the second anchor by a suture extending therebetween.

In addition, or alternatively, to any example disclosed herein, advancing the penetrating member of the inserter instrument into tissue or bone includes advancing the penetrating member through an implantable scaffold positioned against the outer surface of the tissue or bone.

In addition, or alternatively, to any example disclosed herein, deploying the second anchor secures the implantable scaffold against the outer surface of the tissue or bone.

The above summary of some embodiments, aspects, and/or examples is not intended to describe each disclosed embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of an inserter instrument of a tissue anchor system according to the disclosure;

FIGS. 2A-2B schematically illustrate example configurations of an anchor assembly of the tissue anchor system usable with the inserter instrument of FIG. 1;

FIG. 3 is a detailed view illustrating selected aspects of the tissue anchor system, including the anchor assembly of FIGS. 2A-2B disposed within the inserter instrument of FIG. 1;

FIGS. 4-6 schematically illustrate selected aspects of a method of using the tissue anchor system to secure an implantable scaffold against tissue;

FIGS. 7-9 schematically illustrate selected aspects of a method of using the tissue anchor system to secure an implantable scaffold against tissue;

FIGS. 10-12 schematically illustrate selected aspects of a method of using the tissue anchor system to close a tear in tissue;

FIGS. 13-15 schematically illustrate selected aspects of a method of using the tissue anchor system to secure an implantable scaffold against bone;

FIG. 16 is a detailed view illustrating selected aspects of an alternative configuration the inserter instrument of FIG. 1;

FIG. 17 schematically illustrates selected aspects of using the inserter instrument of FIG. 16 in the methods disclosed herein;

FIGS. 18A-18B schematically illustrate selected aspects of an alternative configuration of the anchor assembly of the tissue anchor system;

FIGS. 19A-19B schematically illustrate selected aspects of an alternative configuration of the anchor assembly of the tissue anchor system; and

FIGS. 20A-20B schematically illustrate selected aspects of an alternative configuration of the anchor assembly of the tissue anchor system.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale and/or which may include changes of scale therein. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, enlightened by the present disclosure, would understand desired dimensions, ranges, and/or values may deviate from those expressly disclosed. Additionally, the term “substantially”, when used in reference to two dimensions being substantially equal or the same, shall generally refer to a difference of less than or equal to 5%.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together unless such discrete structures or elements are no longer distinguishable from one another.

It is noted that references in the specification to “an embodiment”, “some embodiments”, “other embodiments”, etc., indicate that the embodiment(s) described may include one or more particular features, structures, and/or characteristics. However, such recitations do not necessarily mean that all embodiments include the particular features, structures, and/or characteristics. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described herein, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein.

The systems, devices, and/or methods disclosed herein may provide a number of desirable features and benefits as described in more detail below.

With its complexity, range of motion, and extensive use, a common soft tissue injury is damage to the rotator cuff or rotator cuff tendons. Damage to the rotator cuff is a potentially serious medical condition that may occur during hyperextension, from an acute traumatic tear, or from overuse of the joint. An accepted treatment for rotator cuff tears may include reattaching the torn tendon to the humeral head using bone anchors. Additionally, in treating rotator cuff tears, an accepted practice may also include the placement of a scaffold over the repaired tendon to mechanically reinforce the repaired tendon and/or to promote tissue reformation. Therefore, there is an ongoing need to efficiently secure medical implants to tissue or bone during an arthroscopic procedure in order to treat injuries to the rotator cuff, rotator cuff tendons, or other soft tissue or tendon injuries throughout a body.

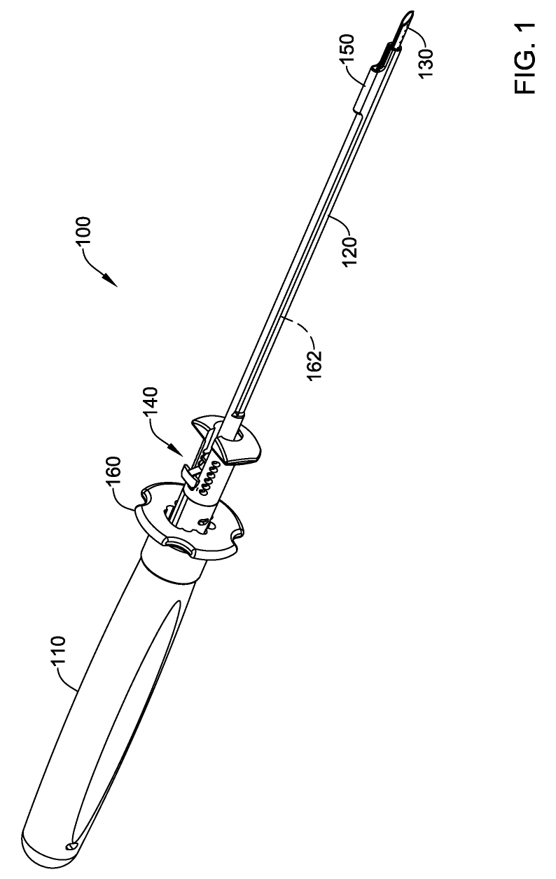

FIG. 1 illustrates selected aspects of an inserter instrument 100 of a tissue anchor system according to the disclosure. The inserter instrument 100 may include a handle 110 disposed proximate a proximal end of the inserter instrument 100. The handle 110 may be configured to be grasped by a hand of a user. The inserter instrument 100 may include an elongate shaft 120 extending distally from the handle 110. The elongate shaft 120 may include a lumen extending therethrough.

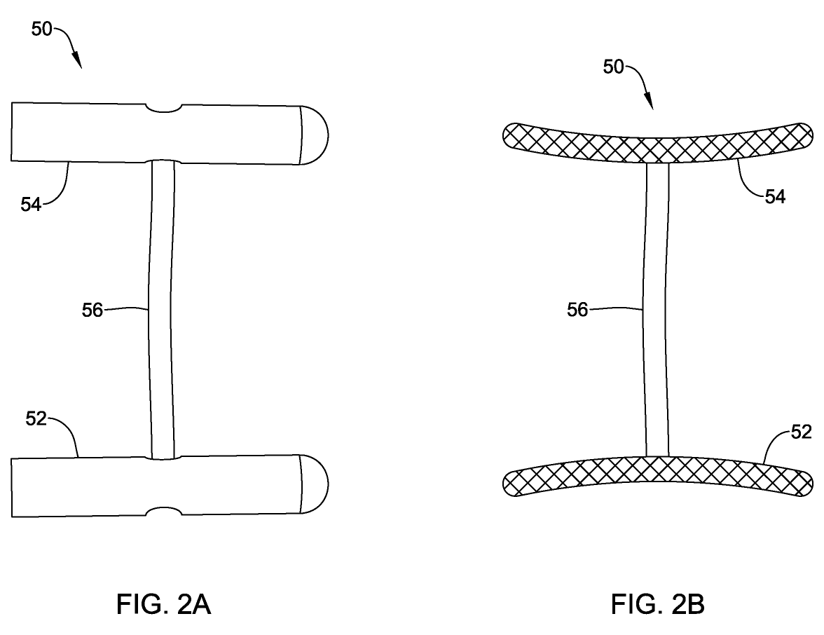

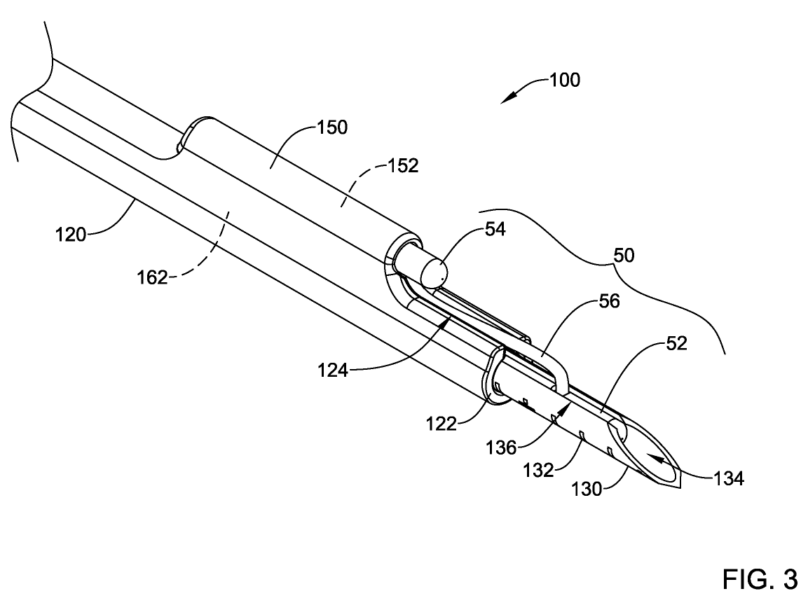

In some embodiments, the inserter instrument 100 may include a penetrating member 130 disposed at a distal end of the inserter instrument 100. In some embodiments, the penetrating member 130 may extend distally from a distal end of the elongate shaft 120 of the inserter instrument 100. In some embodiments, the penetrating member 130 may be axially movable relative to the elongate shaft 120. In some embodiments, the penetrating member 130 may include a plurality of index marks 132 (e.g., FIG. 3) disposed on and/or formed in an outer surface of the penetrating member 130. The penetrating member 130 may include a lumen 134 (e.g., FIG. 3) configured to removably receive a first anchor 52 of an anchor assembly 50 (e.g., FIGS. 2A-3) of the tissue anchor system.

In some embodiments, the inserter instrument 100 may include an adjustment mechanism 140 disposed at and/or proximate a distal end of the handle 110 for adjusting an extension length of the penetrating member 130 beyond the distal end of the elongate shaft 120. Accordingly, the adjustment mechanism 140 may be operably connected to the penetrating member 130. The extension length of the penetrating member 130 may be adjustable to control how deep the penetrating member 130 may extend and/or may be inserted into tissue or bone during a procedure, as discussed herein. In some embodiments, the adjustment mechanism 140 may be a manual adjustment mechanism. In some embodiments, the adjustment mechanism 140 may be an electronic adjustment mechanism. Other configurations are also contemplated.

In some embodiments, the inserter instrument 100 and/or the elongate shaft 120 may include a retainer 150 positioned proximally of and laterally offset from the penetrating member 130. In some embodiments, the retainer 150 may include a lumen 152 (e.g., FIG. 3) configured to removably receive a second anchor 54 of the anchor assembly 50 (e.g., FIGS. 2A-3) of the tissue anchor system. In at least some embodiments, the retainer 150 may be monolithically formed with the elongate shaft 120. In some embodiments, the retainer 150 may be formed from a tubular member that is later fixedly attached to the elongate shaft 120, such as via welding, adhesive bonding, etc.

In some embodiments, the inserter instrument 100 and/or the handle 110 may include a deployment knob 160. In some embodiments, the deployment knob 160 may be movable relative to the handle 110 and/or the elongate shaft 120 to deploy the first anchor 52 of the anchor assembly 50 from the lumen 134 of the penetrating member 130, as discussed herein. In some embodiments, the deployment knob 160 may be axially movable relative to the handle 110, the elongate shaft 120, and/or the penetrating member 130. In some alternative embodiments, the deployment knob 160 may be rotatable relative to the handle 110 and/or the elongate shaft 120. Other configurations are also contemplated.

In some embodiments, the inserter instrument 100 may include a pusher shaft 162 movably disposed within the handle 110, the elongate shaft 120, and/or the penetrating member 130. The deployment knob 160 may be operably connected to the pusher shaft 162. In some embodiments, the pusher shaft 162 may be axially movable relative to the handle 110, the elongate shaft 120, and/or the penetrating member 130. In some embodiments, the pusher shaft 162 may be axially slidable relative to the handle 110, the elongate shaft 120, and/or the penetrating member 130. In some embodiments, the pusher shaft 162 may be threadably engaged with the handle 110, the elongate shaft 120, the penetrating member 130, and/or the deployment knob 160. Other configurations are also contemplated. The deployment knob 160 may be configured to move and/or translate the pusher shaft 162 relative to the handle 110, the elongate shaft 120, and/or the penetrating member 130 to deploy and/or eject the first anchor 52 of the anchor assembly 50 from the lumen 134 of the penetrating member 130.

FIGS. 2A and 2B illustrate example configurations of an anchor assembly 50 of the tissue anchor system. The anchor assembly 50 may include a first anchor 52, a second anchor 54, and a suture 56 extending therebetween. In some embodiments, the suture 56 may include and/or may be formed from or as a thread, a filament, a wire, etc. In at least some embodiments, the suture 56 may be fixedly attached and/or fixedly secured to the first anchor 52 and/or the second anchor 54.

In some embodiments, a first end of the suture 56 may be coupled and/or fixedly attached to the first anchor 52 at a medial location between a first end of the first anchor 52 and a second end of the first anchor 52 disposed opposite the first end of the first anchor 52. In some embodiments, a second end of the suture 56 may be coupled and/or fixedly attached to the second anchor 54 at a medial location between a first end of the second anchor 54 and a second end of the second anchor 54 disposed opposite the first end of the second anchor 54. In some alternative configurations, the first end of the suture 56 may be coupled and/or fixedly attached to the first end of the first anchor 52 or the second end of the first anchor 52. In some alternative configurations, the second end of the suture 56 may be coupled and/or fixedly attached to the first end of the second anchor 54 or the second end of the second anchor 54. Other configurations, including combinations thereof, are also contemplated. In some embodiments, a length of the suture 56 extending between the first anchor 52 and the second anchor 54 may be fixed.

In some embodiments, the first anchor 52 and/or the second anchor 54 may each include and/or may each be formed from an elongate member. In one non-limiting example, the first anchor 52 and/or the second anchor 54 may be formed from a polymeric material. Some suitable but non-limiting examples of materials for the first anchor 52 and/or the second anchor 54, including but not limited to polymeric materials, metallic materials, composite materials, and the like, are discussed below. In some embodiments, the first anchor 52 and/or the second anchor 54 may each be formed from a substantially rigid polymeric material, as seen in FIG. 2A. In some embodiments, the first anchor 52 and/or the second anchor 54 may each be formed from a flexible polymeric material, a woven material, a braided material, etc., as seen in FIG. 2B. Other configurations, including combinations thereof, are also contemplated.

FIG. 3 is a detailed view illustrating selected aspects of the tissue anchor system, including the anchor assembly 50 of FIGS. 2A-2B disposed within the inserter instrument 100 of FIG. 1. In some embodiments, the first anchor 52 of the anchor assembly 50 may be removably and/or slidably disposed within the lumen 134 of the penetrating member 130. In some embodiments, the second anchor 54 of the anchor assembly 50 may be removably and/or slidably disposed within the lumen 154 of the retainer 150. In at least some embodiments, a longitudinal axis of the first anchor 52 when the first anchor 52 is disposed within the lumen 134 of the penetrating member 130 may be parallel to a longitudinal axis of the second anchor 54 when the second anchor 54 is disposed within the lumen 152 of the retainer 150.

In some embodiments, the inserter instrument 100 and/or the elongate shaft 120 may include a distal shoulder 122 disposed at and/or on the distal end of the elongate shaft 120. The distal shoulder 122 may face in a distal direction. In some embodiments, the elongate shaft 120 may include a longitudinal slot 124 extending from the distal shoulder 122 to the retainer 150 and/or the lumen 152 of the retainer 150. In some embodiments, the longitudinal slot 124 of the elongate shaft 120 may be configured to receive at least a portion of the suture 56 therein. In at least some embodiments, the longitudinal slot 124 may extend through a side wall of the elongate shaft 120 such that the lumen of the elongate shaft 120 is in communication with an exterior of the elongate shaft 120 through the side wall of the elongate shaft 120.

The penetrating member 130 may extend distally of the distal shoulder 122 of the elongate shaft 120 to a distal tip. The lumen 134 of the penetrating member 130 may extend proximally from the distal tip of the penetrating member 130. In some embodiments, the penetrating member 130 may include a longitudinal slot 136 extending proximally from the distal tip of the penetrating member 130. In some embodiments, the longitudinal slot 136 of the penetrating member 130 may be configured to receive at least a portion of the suture 56 therein. In at least some embodiments, the longitudinal slot 136 may extend through a side wall of the penetrating member 130 such that the lumen 134 of the penetrating member 130 is in communication with an exterior of the penetrating member 130 and/or the longitudinal slot 124 of the elongate shaft 120 through the side wall of the penetrating member 130.

In some embodiments, the first anchor 52 of the anchor assembly 50 may be deployable from the lumen 134 of the penetrating member 130 into tissue or bone. In some embodiments, the second anchor 54 of the anchor assembly 50 may be deployable from the lumen 152 of the retainer 150 outside of the tissue or bone.

In some embodiments, the penetrating member 130 may be configured to penetrate tissue. In some embodiments, the inserter instrument 100, the pusher shaft 162, and/or the deployment knob 160 (e.g., FIG. 1) may be configured to deploy the first anchor 52 of the anchor assembly 50 from the lumen 134 of the penetrating member 130 below an upper surface of the tissue. In some embodiments, the penetrating member 130 may be configured to penetrate through tissue. In some embodiments, the inserter instrument 100, the pusher shaft 162, and/or the deployment knob 160 (e.g., FIG. 1) may be configured to deploy the first anchor 52 of the anchor assembly 50 from the lumen 134 of the penetrating member 130 below the tissue. In some embodiments, the inserter instrument 100 may be configured to deploy the second anchor 54 of the anchor assembly 50 from the lumen 152 of the retainer 150 above the upper surface of the tissue.

In some embodiments, the penetrating member 130 may be configured to penetrate bone and/or a cortical shell of a bone. In some embodiments, the inserter instrument 100, the pusher shaft 162, and/or the deployment knob 160 (e.g., FIG. 1) may be configured to deploy the first anchor 52 of the anchor assembly 50 from the lumen 134 of the penetrating member 130 below an upper surface of the bone and/or below the cortical shell of the bone. In some embodiments, the inserter instrument 100, the pusher shaft 162, and/or the deployment knob 160 (e.g., FIG. 1) may be configured to deploy the first anchor 52 of the anchor assembly 50 from the lumen 134 of the penetrating member 130 into cancellous bone disposed beneath the cortical shell. In some embodiments, the inserter instrument 100 may be configured to deploy the second anchor 54 of the anchor assembly 50 from the lumen 152 of the retainer 150 above the upper surface of the bone and/or the cortical shell of the bone.

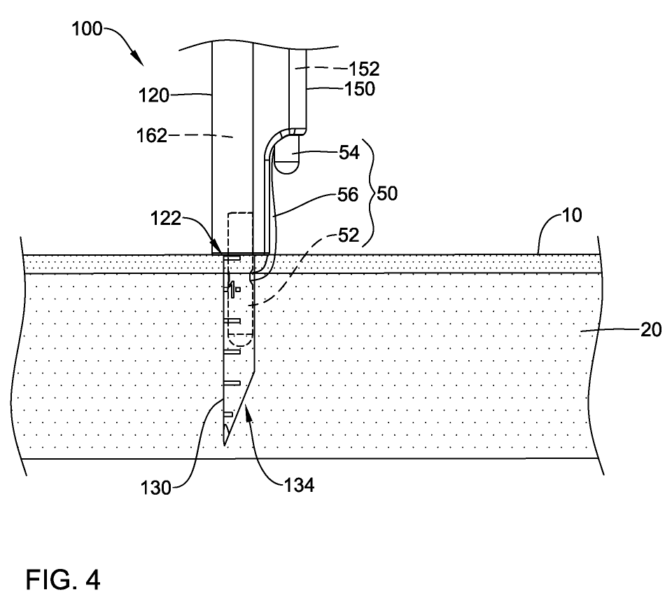

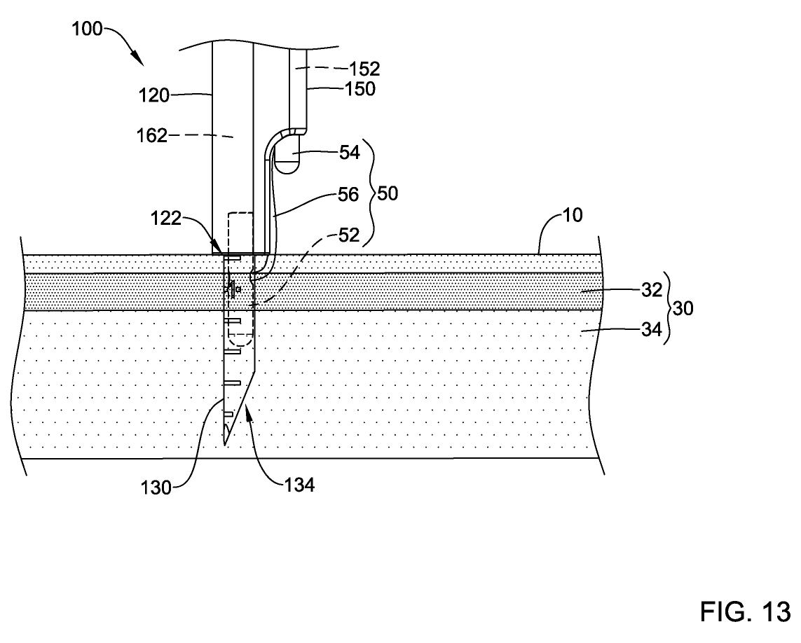

FIGS. 4-15 illustrate selected aspects associated with various methods of deploying the anchor assembly 50 and/or methods of use of the tissue anchor system. In some embodiments, the tissue anchor system may optionally comprise an implantable scaffold 10 configured to be deployed and/or positioned against an outer surface of tissue 20 (e.g., FIGS. 4-9) or bone 30 (e.g., FIGS. 13-15). The implantable scaffold 10 may be a medical implant configured to reinforce the tissue 20 or the bone 30, and/or to promote healing and/or strength within the tissue 20 or the bone 30. In one non-limiting example, the implantable scaffold 10 may include a collagen implant. In another non-limiting example, the implantable scaffold 10 may include a fabric, a mesh, or other reinforcing element. In some embodiments, the implantable scaffold 10 may be treated with, coated with, impregnated with, etc. a therapeutic agent. Other configurations are also contemplated.

FIGS. 4-6 schematically illustrate selected aspects of a method of deploying the anchor assembly 50 wherein the first anchor 52 of the anchor assembly 50 is deployed within the tissue 20. In some embodiments, the method of deploying the anchor assembly 50 may comprise advancing a distal tip of the penetrating member 130 of the inserter instrument 100 into the tissue 20 until the distal shoulder 122 of the inserter instrument 100 and/or the elongate shaft 120 abuts and/or contacts an outer surface of the tissue 20. In some embodiments, advancing the distal tip of the penetrating member 130 of the inserter instrument 100 into the tissue 20 may include advancing the distal tip of the penetrating member 130 through the implantable scaffold 10, as seen in FIG. 4. In such embodiments, the method may comprise advancing the distal tip of the penetrating member 130 of the inserter instrument 100 through the implantable scaffold 10 and into the tissue 20 until the distal shoulder 122 of the inserter instrument 100 and/or the elongate shaft 120 abuts and/or contacts an outer surface of the implantable scaffold 10.

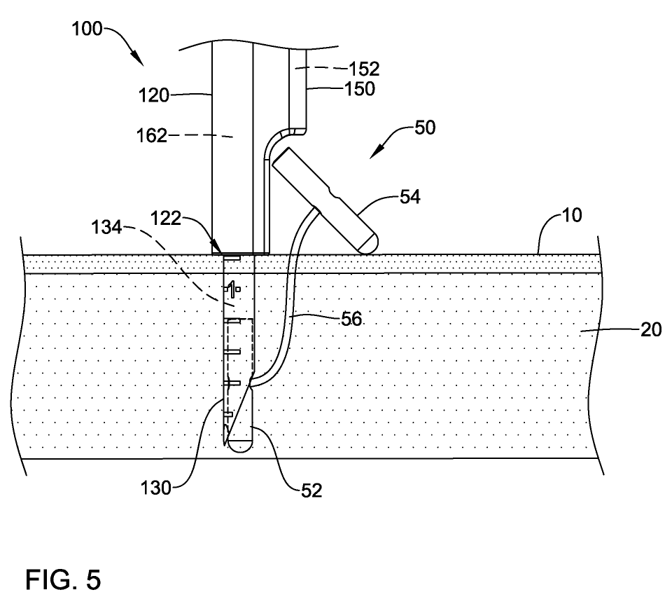

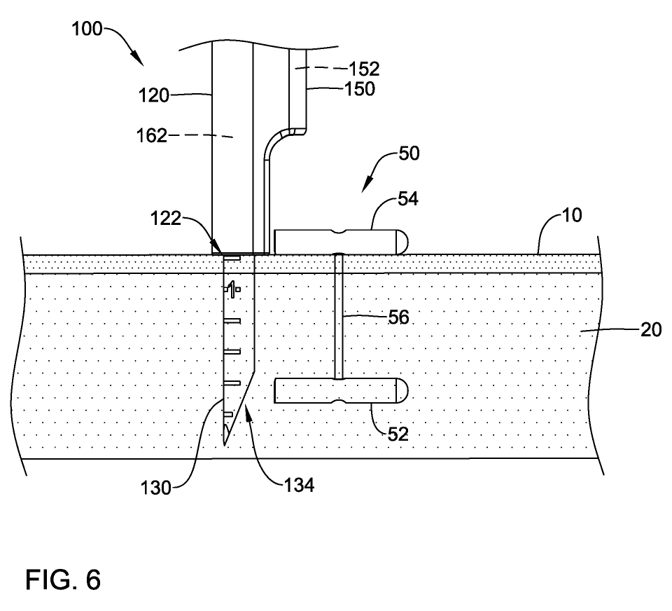

In some embodiments, the method of deploying the anchor assembly 50 may comprise deploying the first anchor 52 of the anchor assembly 50 from the lumen 134 of the penetrating member 130. In some embodiments, deploying the first anchor 52 of the anchor assembly 50 from the lumen 134 of the penetrating member 130 may include deploying the first anchor 52 of the anchor assembly 50 from the lumen 134 of the penetrating member 130 within the tissue 20, as seen in FIGS. 5-6. In some embodiments, deploying the first anchor 52 of the anchor assembly 50 from the lumen 134 of the penetrating member 130 may include advancing the pusher shaft 162 (e.g., FIGS. 1, 3) distally within and/or relative to the elongate shaft 120 and/or the penetrating member 130 to deploy and/or eject the first anchor 52 of the anchor assembly 50 from the lumen 134 of the penetrating member 130.

In some embodiments, the method of deploying the anchor assembly 50 may comprise deploying the second anchor 54 of the anchor assembly 50 from the lumen 152 of the retainer 150 of the inserter instrument 100 outside of the tissue 20 and/or above the outer surface of the tissue 20. In some embodiments, the method of deploying the anchor assembly 50 may comprise deploying the second anchor 54 of the anchor assembly 50 from the lumen 152 of the retainer 150 of the inserter instrument 100 outside of and/or above the outer surface of the implantable scaffold 10, as seen in FIG. 6. In some embodiments, after deploying the second anchor 54 of the anchor assembly 50 from the lumen 152 of the retainer 150, the second anchor 54 may lay on top of and/or against the outer surface of the implantable scaffold 10. In some embodiments, the second anchor 54 of the anchor assembly 50 may be configured to secure the implantable scaffold 10 against the outer surface of the tissue 20. In some embodiments, deploying the second anchor 54 of the anchor assembly 50 may secure the implantable scaffold 10 against the outer surface of the tissue 20.

In some embodiments, the method of deploying the anchor assembly 50 may comprise, prior to advancing the distal tip of the penetrating member 130 of the inserter instrument 100 into the tissue 20, adjusting the extension length of the penetrating member 130 beyond the distal end of the elongate shaft 120 and/or the distal shoulder 122 of the inserter instrument 100 and/or the elongate shaft 120, using the adjustment mechanism 140 for example. In some embodiments, the extension length of the penetrating member 130 may be adjusted to control a depth of deployment of the first anchor 52 of the anchor assembly 50 within the tissue 20, below the outer surface of the tissue 20, and/or below the outer surface of the implantable scaffold 10. In at least some embodiments, the extension length of the penetrating member 130 may be adjusted and/or set such that the distal tip of the penetrating member 130 does not and/or cannot penetrate completely through the tissue 20 when the distal shoulder 122 of the inserter instrument 100 and/or the elongate shaft 120 abuts the outer surface of the tissue 20 and/or the outer surface of the implantable scaffold 10.

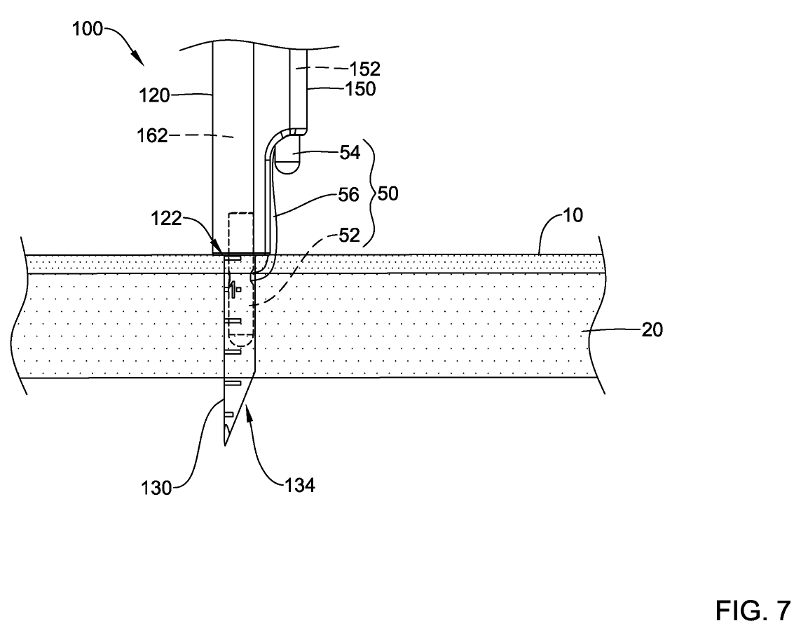

FIGS. 7-9 schematically illustrate selected aspects of a method of deploying the anchor assembly 50 wherein the first anchor 52 of the anchor assembly 50 is deployed through and/or below the tissue 20. In some embodiments, the method of deploying the anchor assembly 50 may comprise advancing a distal tip of the penetrating member 130 of the inserter instrument 100 into the tissue 20 until the distal shoulder 122 of the inserter instrument 100 and/or the elongate shaft 120 abuts and/or contacts an outer surface of the tissue 20. In some embodiments, advancing the distal tip of the penetrating member 130 of the inserter instrument 100 into the tissue 20 may include advancing the distal tip of the penetrating member 130 through the tissue 20 such that the distal tip of the penetrating member 130 is disposed below the tissue 20 when the distal shoulder 122 of the inserter instrument 100 and/or the elongate shaft 120 abuts and/or contacts the outer surface of the tissue 20.

In some embodiments, advancing the distal tip of the penetrating member 130 of the inserter instrument 100 into the tissue 20 may include advancing the distal tip of the penetrating member 130 through the implantable scaffold 10, as seen in FIG. 7. In such embodiments, the method may comprise advancing the distal tip of the penetrating member 130 of the inserter instrument 100 through the implantable scaffold 10 and the tissue 20 until the distal shoulder 122 of the inserter instrument 100 and/or the elongate shaft 120 abuts and/or contacts an outer surface of the implantable scaffold 10 such that the distal tip of the penetrating member 130 is disposed below the tissue 20 when the distal shoulder 122 of the inserter instrument 100 and/or the elongate shaft 120 abuts and/or contacts the outer surface of the implantable scaffold 10.

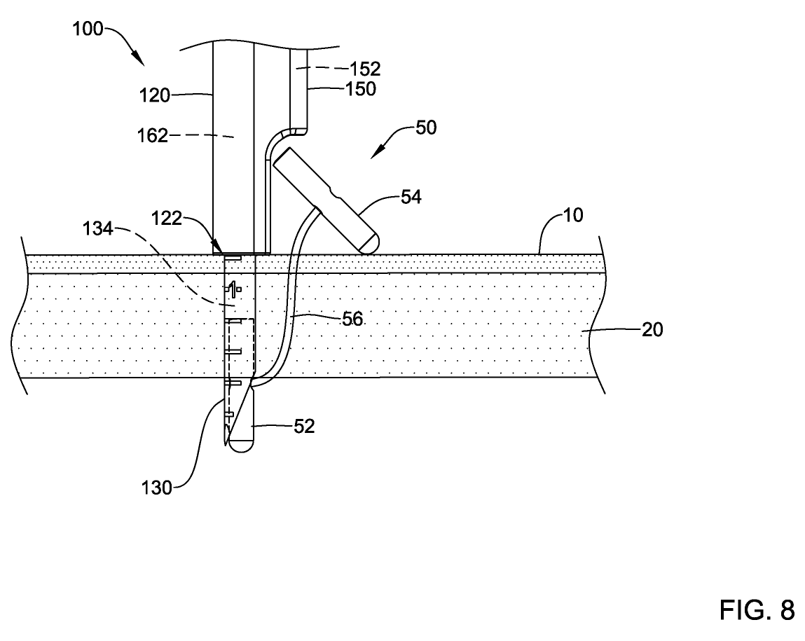

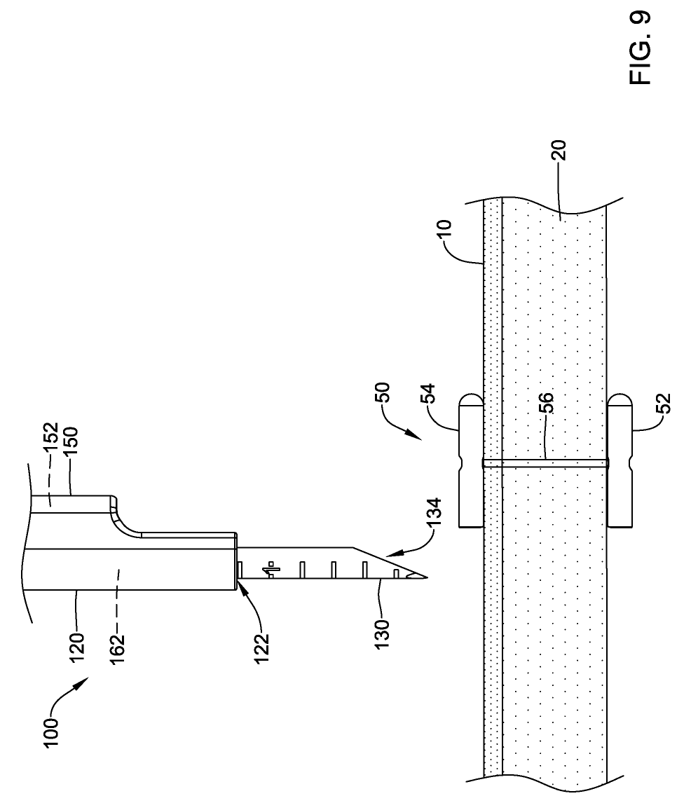

In some embodiments, the method of deploying the anchor assembly 50 may comprise deploying the first anchor 52 of the anchor assembly 50 from the lumen 134 of the penetrating member 130. In some embodiments, deploying the first anchor 52 of the anchor assembly 50 from the lumen 134 of the penetrating member 130 may include deploying the first anchor 52 of the anchor assembly 50 from the lumen 134 of the penetrating member 130 below the tissue 20, as seen in FIGS. 8-9. In some embodiments, deploying the first anchor 52 of the anchor assembly 50 from the lumen 134 of the penetrating member 130 may include advancing the pusher shaft 162 (e.g., FIGS. 1, 3) distally within and/or relative to the elongate shaft 120 and/or the penetrating member 130 to deploy and/or eject the first anchor 52 of the anchor assembly 50 from the lumen 134 of the penetrating member 130.

In some embodiments, the method of deploying the anchor assembly 50 may comprise deploying the second anchor 54 of the anchor assembly 50 from the lumen 152 of the retainer 150 of the inserter instrument 100 outside of the tissue 20 and/or above the outer surface of the tissue 20. In some embodiments, the method of deploying the anchor assembly 50 may comprise deploying the second anchor 54 of the anchor assembly 50 from the lumen 152 of the retainer 150 of the inserter instrument 100 outside of and/or above the outer surface of the implantable scaffold 10, as seen in FIG. 9. In some embodiments, after deploying the second anchor 54 of the anchor assembly 50 from the lumen 152 of the retainer 150, the second anchor 54 may lay on top of and/or against the outer surface of the implantable scaffold 10. In some embodiments, deploying the second anchor 54 of the anchor assembly 50 may secure the implantable scaffold 10 against the outer surface of the tissue 20.

In some embodiments, the method of deploying the anchor assembly 50 may comprise, prior to advancing the distal tip of the penetrating member 130 of the inserter instrument 100 into the tissue 20, adjusting the extension length of the penetrating member 130 beyond the distal end of the elongate shaft 120 and/or the distal shoulder 122 of the inserter instrument 100 and/or the elongate shaft 120, using the adjustment mechanism 140 for example. In some embodiments, the extension length of the penetrating member 130 may be adjusted to control a depth of deployment of the first anchor 52 of the anchor assembly 50 below the tissue 20. In at least some embodiments, the extension length of the penetrating member 130 may be adjusted and/or set such that the distal tip of the penetrating member 130 penetrates completely through the tissue 20 when the distal shoulder 122 of the inserter instrument 100 and/or the elongate shaft 120 abuts the outer surface of the tissue 20 and/or the outer surface of the implantable scaffold 10.

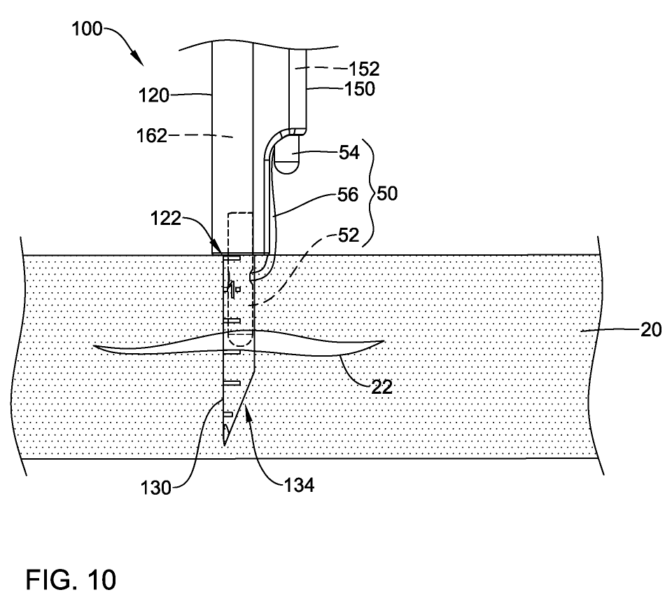

FIGS. 10-12 schematically illustrate selected aspects of a method of deploying the anchor assembly 50 wherein the first anchor 52 of the anchor assembly 50 is deployed within the tissue 20 to close a tear 22 in the tissue 20. In some embodiments, the method of deploying the anchor assembly 50 may comprise advancing a distal tip of the penetrating member 130 of the inserter instrument 100 into the tissue 20 and across the tear 22 until the distal shoulder 122 of the inserter instrument 100 and/or the elongate shaft 120 abuts and/or contacts an outer surface of the tissue 20, as seen in FIG. 10. In some embodiments, advancing the distal tip of the penetrating member 130 of the inserter instrument 100 into the tissue 20 may include advancing the distal tip of the penetrating member 130 through the implantable scaffold 10 (not shown in FIGS. 10-12, but may be included and/or used as in FIGS. 4-9 above). In such embodiments, the method may comprise advancing the distal tip of the penetrating member 130 of the inserter instrument 100 through the implantable scaffold 10 and into the tissue 20 until the distal shoulder 122 of the inserter instrument 100 and/or the elongate shaft 120 abuts and/or contacts an outer surface of the implantable scaffold 10 and the penetrating member 130 extends across the tear 22 in the tissue 20.

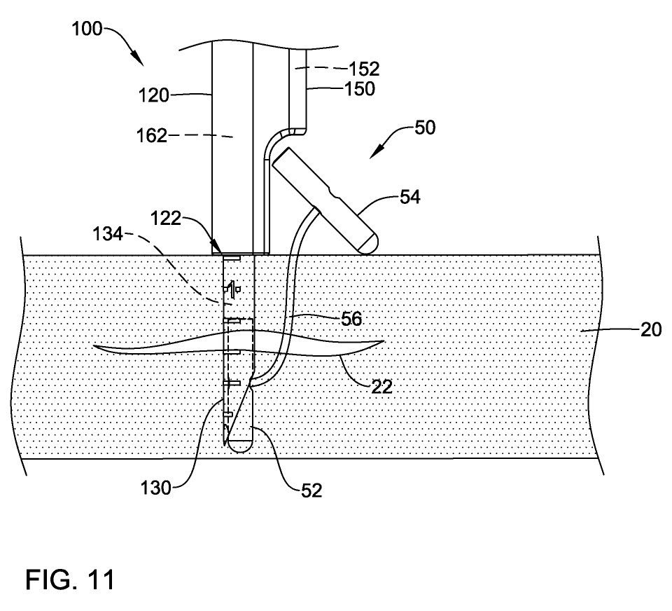

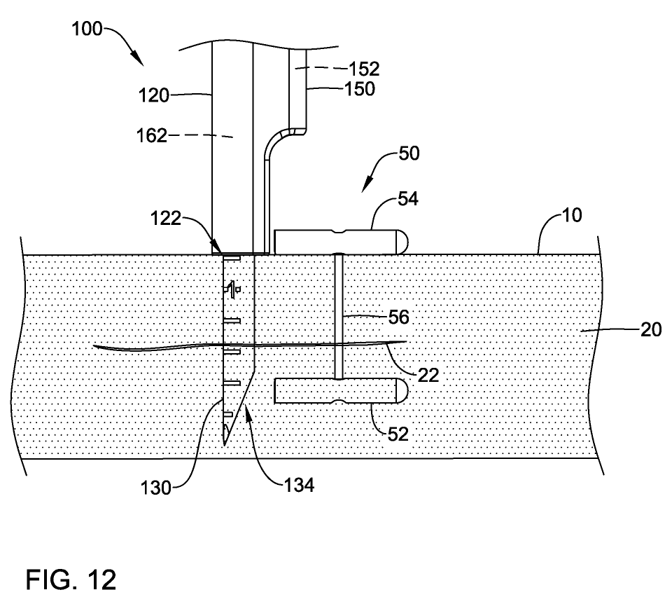

In some embodiments, the method of deploying the anchor assembly 50 may comprise deploying the first anchor 52 of the anchor assembly 50 from the lumen 134 of the penetrating member 130. In some embodiments, deploying the first anchor 52 of the anchor assembly 50 from the lumen 134 of the penetrating member 130 may include deploying the first anchor 52 of the anchor assembly 50 from the lumen 134 of the penetrating member 130 within the tissue 20 on a distal side of the tear 22, as seen in FIGS. 11-12. In some embodiments, deploying the first anchor 52 of the anchor assembly 50 from the lumen 134 of the penetrating member 130 may include advancing the pusher shaft 162 (e.g., FIGS. 1, 3) distally within and/or relative to the elongate shaft 120 and/or the penetrating member 130 to deploy and/or eject the first anchor 52 of the anchor assembly 50 from the lumen 134 of the penetrating member 130 on the distal side of the tear 22.

In some embodiments, the method of deploying the anchor assembly 50 may comprise deploying the second anchor 54 of the anchor assembly 50 from the lumen 152 of the retainer 150 of the inserter instrument 100 outside of the tissue 20, above the outer surface of the tissue 20, and/or proximal of the tear 22, as seen in FIG. 12. In some embodiments, the method of deploying the anchor assembly 50 may comprise deploying the second anchor 54 of the anchor assembly 50 from the lumen 152 of the retainer 150 of the inserter instrument 100 outside of and/or above the outer surface of the implantable scaffold 10. In some embodiments, after deploying the second anchor 54 of the anchor assembly 50 from the lumen 152 of the retainer 150, the second anchor 54 may lay on top of and/or against the outer surface of the implantable scaffold 10. In some embodiments, the second anchor 54 of the anchor assembly 50 may be configured to secure the implantable scaffold 10 against the outer surface of the tissue 20. In some embodiments, deploying the second anchor 54 of the anchor assembly 50 may secure the implantable scaffold 10 against the outer surface of the tissue 20.

In some embodiments, the method of deploying the anchor assembly 50 may comprise, prior to advancing the distal tip of the penetrating member 130 of the inserter instrument 100 into the tissue 20, adjusting the extension length of the penetrating member 130 beyond the distal end of the elongate shaft 120 and/or the distal shoulder 122 of the inserter instrument 100 and/or the elongate shaft 120, using the adjustment mechanism 140 for example. In some embodiments, the extension length of the penetrating member 130 may be adjusted to control a depth of deployment of the first anchor 52 of the anchor assembly 50 within the tissue 20, below the outer surface of the tissue 20, and/or below the outer surface of the implantable scaffold 10 such that the distal tip of the penetrating member 130 extends distal of the tear 22. In at least some embodiments, the extension length of the penetrating member 130 may be adjusted and/or set such that the distal tip of the penetrating member 130 does not and/or cannot penetrate completely through the tissue 20 when the distal shoulder 122 of the inserter instrument 100 and/or the elongate shaft 120 abuts the outer surface of the tissue 20 and/or the outer surface of the implantable scaffold 10.

While not expressly illustrated, in some embodiments, advancing the distal tip of the penetrating member 130 of the inserter instrument 100 into the tissue 20 may include advancing the distal tip of the penetrating member 130 through the tissue 20 such that the distal tip of the penetrating member 130 is disposed below the tissue 20 when the distal shoulder 122 of the inserter instrument 100 and/or the elongate shaft 120 abuts and/or contacts the outer surface of the tissue 20 and/or the outer surface of the implantable scaffold 10, similar to the aspects described and/or shown with respect to FIGS. 7-9 above.

FIGS. 13-15 schematically illustrate selected aspects of a method of deploying the anchor assembly 50 wherein the first anchor 52 of the anchor assembly 50 is deployed within the bone 30 and/or cancellous bone 34 of the bone 30 disposed beneath the cortical shell 32 of the bone 30. As such, the penetrating member 130 may be configured to penetrate the cortical shell 32 of the bone 30. In some embodiments, the method of deploying the anchor assembly 50 may comprise advancing a distal tip of the penetrating member 130 of the inserter instrument 100 into the bone 30 until the distal shoulder 122 of the inserter instrument 100 and/or the elongate shaft 120 abuts and/or contacts an outer surface of the bone 30 and/or the cortical shell 32 of the bone 30. In some embodiments, advancing the distal tip of the penetrating member 130 of the inserter instrument 100 into the tissue 20 may include advancing the distal tip of the penetrating member 130 through the implantable scaffold 10, as seen in FIG. 13. In such embodiments, the method may comprise advancing the distal tip of the penetrating member 130 of the inserter instrument 100 through the implantable scaffold 10 and into the bone 30 until the distal shoulder 122 of the inserter instrument 100 and/or the elongate shaft 120 abuts and/or contacts an outer surface of the implantable scaffold 10.

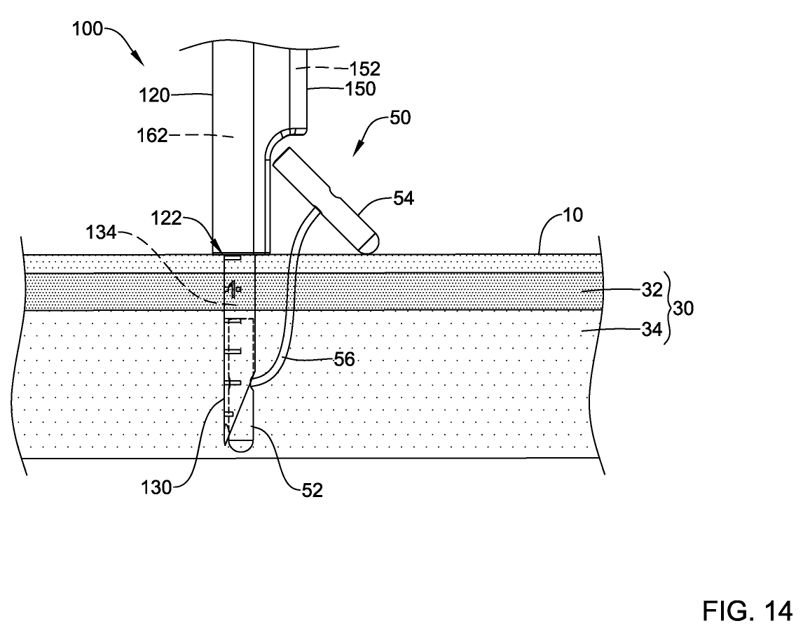

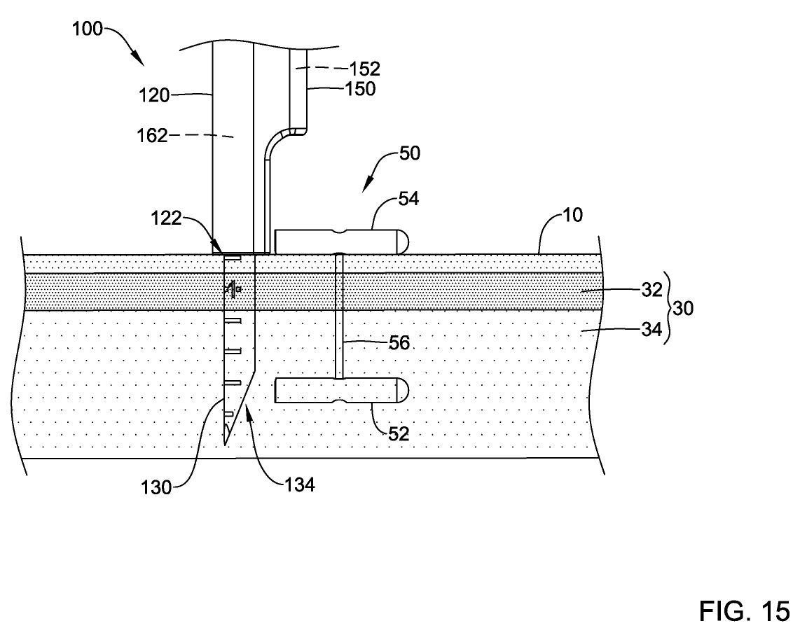

In some embodiments, the method of deploying the anchor assembly 50 may comprise deploying the first anchor 52 of the anchor assembly 50 from the lumen 134 of the penetrating member 130. In some embodiments, deploying the first anchor 52 of the anchor assembly 50 from the lumen 134 of the penetrating member 130 may include deploying the first anchor 52 of the anchor assembly 50 from the lumen 134 of the penetrating member 130 within the cancellous bone 34 of the bone 30 disposed beneath the cortical shell 32 of the bone 30, as seen in FIGS. 14-15. In some embodiments, deploying the first anchor 52 of the anchor assembly 50 from the lumen 134 of the penetrating member 130 may include advancing the pusher shaft 162 (e.g., FIGS. 1, 3) distally within and/or relative to the elongate shaft 120 and/or the penetrating member 130 to deploy and/or eject the first anchor 52 of the anchor assembly 50 from the lumen 134 of the penetrating member 130.

In some embodiments, the method of deploying the anchor assembly 50 may comprise deploying the second anchor 54 of the anchor assembly 50 from the lumen 152 of the retainer 150 of the inserter instrument 100 outside of the bone 30 and/or above the outer surface of the bone 30 and/or the cortical shell 32. In some embodiments, the method of deploying the anchor assembly 50 may comprise deploying the second anchor 54 of the anchor assembly 50 from the lumen 152 of the retainer 150 of the inserter instrument 100 outside of and/or above the outer surface of the implantable scaffold 10, as seen in FIG. 15. In some embodiments, after deploying the second anchor 54 of the anchor assembly 50 from the lumen 152 of the retainer 150, the second anchor 54 may lay on top of and/or against the outer surface of the implantable scaffold 10. In some embodiments, the second anchor 54 of the anchor assembly 50 may be configured to secure the implantable scaffold 10 against the outer surface of the bone 30 and/or the cortical shell 32. In some embodiments, deploying the second anchor 54 of the anchor assembly 50 may secure the implantable scaffold 10 against the outer surface of the bone 30 and/or the cortical shell 32.

In some embodiments, the method of deploying the anchor assembly 50 may comprise, prior to advancing the distal tip of the penetrating member 130 of the inserter instrument 100 into the tissue 20, adjusting the extension length of the penetrating member 130 beyond the distal end of the elongate shaft 120 and/or the distal shoulder 122 of the inserter instrument 100 and/or the elongate shaft 120, using the adjustment mechanism 140 for example. In some embodiments, the extension length of the penetrating member 130 may be adjusted to control a depth of deployment of the first anchor 52 of the anchor assembly 50 within the bone 30 and/or the cancellous bone 34, below the outer surface of the bone 30 and/or the cortical shell 32, and/or below the outer surface of the implantable scaffold 10. In at least some embodiments, the extension length of the penetrating member 130 may be adjusted and/or set such that the distal tip of the penetrating member 130 penetrates completely through the cortical shell 32 when the distal shoulder 122 of the inserter instrument 100 and/or the elongate shaft 120 abuts the outer surface of the tissue 20 and/or the outer surface of the implantable scaffold 10.

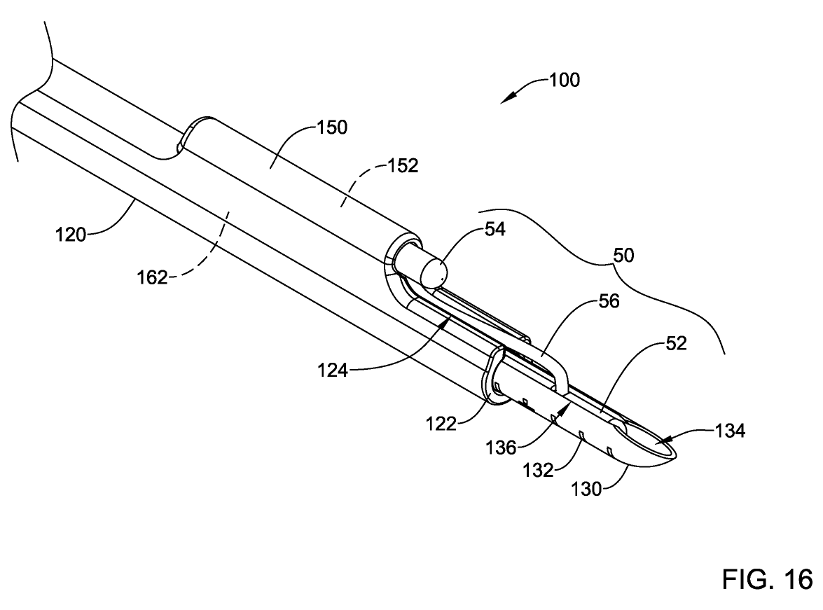

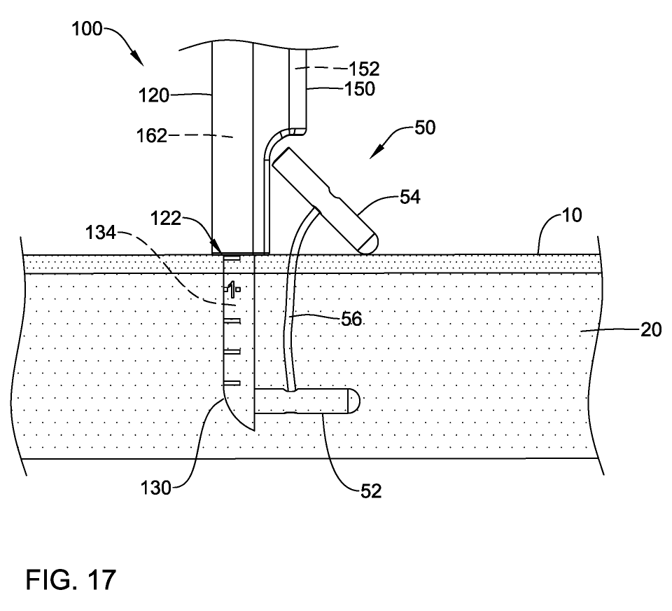

FIG. 16 illustrates selected aspects of an alternative configuration the inserter instrument of FIG. 1 including selected aspects of an example configuration of the penetrating member 130. In some embodiments, the penetrating member 130 may include a curved distal tip opening laterally from a longitudinal axis of the lumen 134 of the penetrating member 130 and/or the lumen of the elongate shaft 120. FIG. 17 schematically illustrates selected aspects of using the inserter instrument of FIG. 16 in the methods disclosed herein. In some embodiments, the first anchor 52 of the anchor assembly 50 may be deployable from the lumen 134 of the penetrating member 130 in a direction non-parallel to the longitudinal axis of the first anchor 52 of the anchor assembly 50 when the first anchor 52 of the anchor assembly 50 is disposed within the lumen 134 of the penetrating member 130. For example, the first anchor 52 may be deployable in a lateral direction from the penetrating member 130. Other configurations are also contemplated.

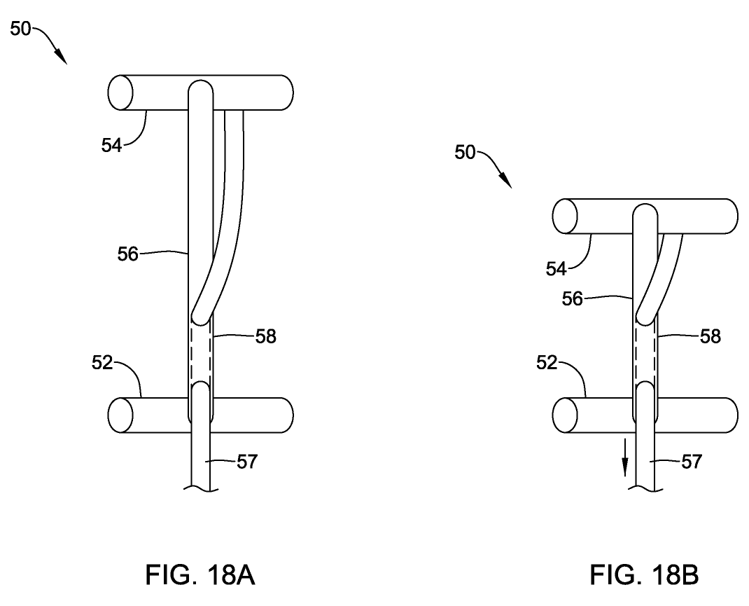

FIGS. 18A-20B schematically illustrate selected aspects of alternative configurations of the anchor assembly 50 of the tissue anchor system described herein. In some embodiments, the length of the suture 56 extending between the first anchor 52 and the second anchor 54 of the anchor assembly 50 may be adjustable. In some embodiments, the suture 56 may be configured to pass through and/or may translate relative to the first anchor 52 of the anchor assembly 50. In some embodiments, the suture 56 may be configured to pass through and/or may translate relative to the second anchor 54 of the anchor assembly 50. In some embodiments, the suture 56 may be configured to pass through and/or may translate relative to the first anchor 52 of the anchor assembly 50, and the suture 56 may be fixed to the second anchor 54 of the anchor assembly 50. In some embodiments, the suture 56 may be configured to pass through and/or may translate relative to the second anchor 54 of the anchor assembly 50, and the suture 56 may be fixed to the first anchor 52 of the anchor assembly 50.

In some embodiments, the length of the suture 56 extending between the first anchor 52 and the second anchor 54 of the anchor assembly 50 may be adjustable via passing a first segment 57 of the suture 56 through and/or within an interior of a second segment 58 of the suture 56, as seen in FIGS. 18A-18B. In some embodiments, tension applied to the first segment 57 of the suture 56 may be configured to shorten and/or reduce the length of the suture 56 extending between the first anchor 52 and the second anchor 54 of the anchor assembly 50. Other configurations are also contemplated.

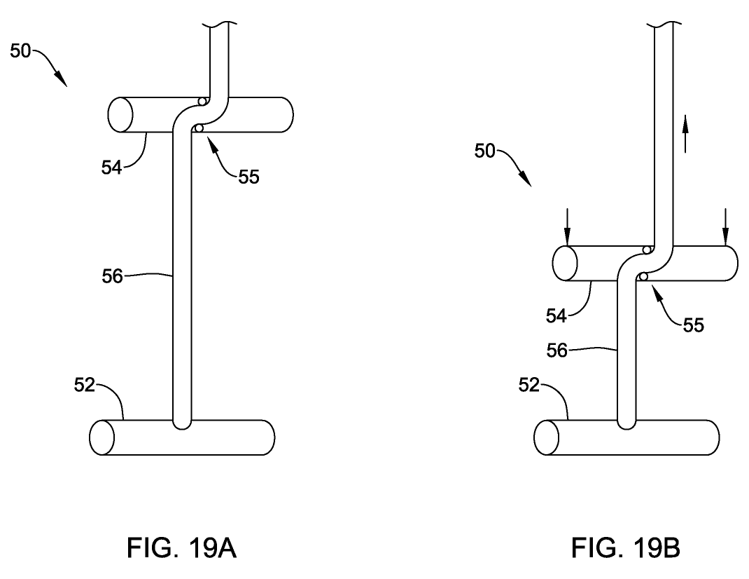

In some embodiments, one of the first anchor 52 and the second anchor 54 may include a one-way locking mechanism 55 formed therein and/or attached thereto. The one-way locking mechanism 55 may be either passive or active. The one-way locking mechanism 55 may be configured to permit translation of the suture 56 relative to the one-way locking mechanism 55 in one direction, and to prevent translation of the suture 56 relative to the one-way locking mechanism 55 in an opposite direction. In the non-limiting example shown FIGS. 19A-19B, the one-way locking mechanism 55 is formed in and/or attached to the second anchor 54, and the following discussion reflects that example. The skilled person will recognize that the features and/or elements may be reversed. In some embodiments, the length of the suture 56 extending between the first anchor 52 and the second anchor 54 of the anchor assembly 50 may be adjustable via passing the suture 56 through and/or within the one-way locking mechanism 55. In some embodiments, tension applied to a free end of the suture 56 and/or a force applied to the second anchor 54 (e.g., whichever anchor includes the one-way locking mechanism 55) toward the first anchor 52, or a force applied to the first anchor 52 toward the second anchor 54 if the first anchor 52 includes the one-way locking mechanism 55, may be configured to shorten and/or reduce the length of the suture 56 extending between the first anchor 52 and the second anchor 54 of the anchor assembly 50. Other configurations are also contemplated.

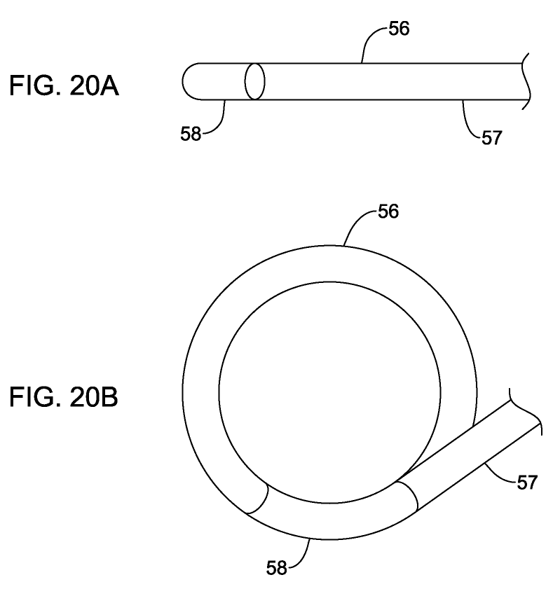

In some embodiments, the anchor assembly 50 may include the suture 56, or a suture-like construct, that passes back through itself to form and/or to create an ever-tightening noose (e.g., a circle, a loop, etc.). In some embodiments, a free end of the suture 56 and/or a first segment 57 of the suture 56 may be passed through a second segment 58 of the suture 56 disposed at an opposing end of the suture 56, as seen in FIGS. 20A-20B. In some embodiments, the second segment 58 of the suture 56 may include a tunnel having a lumen extending therethrough. In some embodiments, the second segment 58 of the suture 56 may include and/or define a trap feature that may permit the first segment 57 of the suture 56 to pass therethrough in a first direction to tighten the noose without passing back through the trap feature in a second direction opposite the first direction (e.g., without loosening). Other configurations are also contemplated.

The materials that can be used for the various components of the system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the tissue anchor system, the inserter instrument, anchor assembly, the anchor(s), the suture, etc. and/or elements or components thereof.

In some embodiments, the system and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA, for example PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, acrylonitrile butadiene styrene (ABS), epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP).

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In some embodiments, portions or all of the system and/or components thereof may be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids a user in determining the location of the system. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-protein and/or anti-bacterial agents (such as 2-methacryroyloxyethyl phosphorylcholine (MPC) and its polymers or copolymers); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure’s scope is, of course, defined in the language in which the appended claims are expressed.

What is claimed is:

1. A tissue anchor system, comprising:
an anchor assembly including a first anchor, a second anchor, and a suture extending therebetween; and
an inserter instrument including a penetrating member and a retainer positioned proximally of and laterally offset from the penetrating member;
wherein the penetrating member includes a lumen configured to removably receive the first anchor;
wherein the retainer includes a lumen configured to removably receive the second anchor.

2. The tissue anchor system of claim 1, wherein the penetrating member includes a curved distal tip.

3. The tissue anchor system of claim 1, wherein the penetrating member is configured to penetrate tissue.

4. The tissue anchor system of claim 3, wherein the inserter instrument is configured to deploy the first anchor from the lumen of the penetrating member below an upper surface of the tissue.

5. The tissue anchor system of claim 3, wherein the inserter instrument is configured to deploy the second anchor from the lumen of the retainer above an upper surface of the tissue.

6. The tissue anchor system of claim 1, wherein the penetrating member is configured to penetrate a cortical shell of a bone.

7. The tissue anchor system of claim 6, wherein the inserter instrument is configured to deploy the first anchor from the lumen of the penetrating member into cancellous bone disposed beneath the cortical shell.

8. The tissue anchor system of claim 6, wherein the inserter instrument is configured to deploy the second anchor from the lumen of the retainer above an upper surface of the cortical shell.

9. The tissue anchor system of claim 1, wherein a length of the suture extending between the first anchor and the second anchor is adjustable.

10. The tissue anchor system of claim 9, wherein the length of the suture extending between the first anchor and the second anchor is adjustable via passing a first segment of the suture through a second segment of the suture.

11. The tissue anchor system of claim 1, wherein the first anchor and the second anchor are each formed from a substantially rigid polymeric material.

12. The tissue anchor system of claim 1, wherein the first anchor and the second anchor are each formed from a woven material or a braided material.

13. A tissue anchor system, comprising:
an anchor assembly including a first anchor, a second anchor, and a suture extending therebetween; and
an inserter instrument including a penetrating member and a retainer positioned proximally of and laterally offset from the penetrating member;
wherein the first anchor is removably disposed within a lumen of the penetrating member;
wherein the second anchor is removably disposed within a lumen of the retainer;
wherein a longitudinal axis of the first anchor when disposed within the lumen of the penetrating member is parallel to a longitudinal axis of the second anchor when disposed within the lumen of the retainer.

14. The tissue anchor system of claim 13, wherein the first anchor is deployable from the lumen of the penetrating member into tissue or bone, and the second anchor is deployable from the lumen of the retainer outside of the tissue or bone.

15. The tissue anchor system of claim 14, further comprising an implantable scaffold configured to be deployed against an outer surface of the tissue or bone.

16. The tissue anchor system of claim 15, wherein the second anchor is configured to secure an implantable scaffold against the outer surface of the tissue or bone.

17. The tissue anchor system of claim 13, wherein a first end of the suture is coupled to the first anchor at a medial location between a first end of the first anchor and a second end of the first anchor, and a second end of the suture is coupled to the second anchor at a medial location between a first end of the second anchor and a second end of the second anchor.

18. The tissue anchor system of claim 13, wherein the first anchor is deployable from the lumen of the penetrator in a direction non-parallel to the longitudinal axis of the first anchor when disposed within the lumen of the penetrating member.

19. A method of deploying an anchor assembly, comprising:
advancing a penetrating member of an inserter instrument into tissue or bone until a distal shoulder of the inserter instrument abuts an outer surface of the tissue or bone;
deploying a first anchor from a lumen of the penetrating member; and
deploying a second anchor from a lumen of a retainer of the inserter instrument outside of the tissue or bone, wherein the lumen of the retainer is disposed proximally of and laterally offset from the penetrating member;
wherein the first anchor is coupled to the second anchor by a suture extending therebetween.

20. The method of claim 19, wherein advancing the penetrating member of the inserter instrument into tissue or bone includes advancing the penetrating member through an implantable scaffold positioned against the outer surface of the tissue or bone; and
wherein deploying the second anchor secures the implantable scaffold against the outer surface of the tissue or bone.
